(19) United States
(12) Patent Application Publication
(10) Pub. No.: US 2009/0017115 A1
(43) Pub. Date: Jan. 15, 2009

(54) REMOVAL OF MATERIAL FROM A PREFORMED TABLET

(76) Inventors: Allan S. Kaplan, Boca Raton, FL (US); Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/814,240

(22) PCT Filed: Jan. 19, 2006
(86) PCT No.: PCT/US2006/001777

§ 371 Date: Sep. 17, 2008

Related U.S. Application Data
(60) Provisional application No. 60/644,939, filed on Jan. 19, 2005.

(51) Int. Cl. B26D 3/08; A61J 3/10; A61K 9/20; A61K 9/44

(52) U.S. Cl. 424/467; 83/879; 83/880; 83/412; 83/409

(57) ABSTRACT

A system and method of removing material from pharmaceutical tablets, utilizing mechanical or laser energy and a conveyance, to produce a score or other defect in said tablet.

### CROSS-REFERENCE TO RELATED APPLICATIONS

This is a Section 371 national filing from US PCT Application Ser. No. PCT/US06/01777, filed 19 Jan. 2006, which claims priority to US Provisional Application, Ser. No. 60/644,939, filed Jan. 19, 2005.

### FIELD OF THE INVENTION

This invention relates to removal of material from preformed tablets, preferably pharmaceutical tablets, such as to create a score in a face of the tablet to facilitate optional tablet breaking.

### BACKGROUND OF THE INVENTION

Many compressed pharmaceutical tablets are manufactured to have a groove, generally known as a score, formed into one or more faces of a tablet during tablet compression. A score is intended to facilitate breaking of the tablet into smaller units by a patient, nurse, aide, pharmacist, and the like. The score both identifies a desired breaking region and provides an indentation into which a sharp edge, such as a knife or razor blade, may be placed to aid the breaking process. A score also may weaken a tablet so that less force is required to break the tablet than would be required if no score were provided and leads the tablet to break at the desired place (i.e., the score region) when manual force is applied to the sides of the tablet apart from the score so that the tablet tends to break through the score region.

In the current art, scoring of compressed tablets is typically performed during tablet compression, utilizing an embossing that is part of or integral with an upper or lower tablet punch. Alternatively, and less commonly, there are examples of vertical scores that are formed from a flange or embossing extending vertically from the tablet die so that they score the tablet vertically as it is produced in the die.

Conventional scoring processes have several disadvantages. For example, the design of scores is limited with regard to shape, size and depth for reasons that include, without limitation, the preference for non-acute angles and smooth contours in the embossing to eliminate or minimize unwanted sticking of material to the embossing rather than to the tablet—a condition known in the pharmaceutical tabletting arts as “picking.”

Another disadvantage of conventional scoring is the limitation of its location either to the top and/or bottom face of the tablet (as oriented in the die) caused by embossing on the corresponding tablet punch(es), or to the side face of the tablet (as oriented in the die) caused by vertically-oriented embossing (vertically-oriented protrusion) into the space contained within the tablet die. Any such embossing or protrusion into the die that is other than vertical will block egress of the intact compressed tablet from the die. Placement of a score in a position other than in a top or bottom face, or vertically on a side of the tablet cannot be performed coincident with tablet compression according to current production methods while yielding a tablet than can be ejected from the die in which compression occurs without damaging the tablet.

A further disadvantage of conventional scoring using a tablet press is that the material compressed below a conventional score that is produced by the embossing found on a top punch or bottom punch (“below” in this sentence assumes herein that the tablet is oriented so that the score is at the top of the tablet as it may exist not necessarily in the die but rather on, for example, a countertop or other flat surface) tends to have an increased hardness as compared to the remainder of the tablet, due to relative over-compression of that section of the tablet in vertical register with the embossing. This condition may lead to stress within the material near the score, which can induce more uneven, irregular, chip-prone tablet breaking than would be the case if the same tablet were broken elsewhere than through the score.

Treatment of compressed tablets after their removal from the compression dies has generally been limited to application of coatings (and subsequent dosage form modification such as laser drilling for controlled release purposes) or printing of indicia on the tablet surface(s). U.S. Pat. No. 5,894,801 issued Apr. 20, 1999 to Ackley discloses dispensing pellet-shaped objects such as tablets onto a conveyer, sensing the objects, rectifying them on the basis of said sensing to orient them correctly and then drilling a hole via laser into their coating but only into the coating, not into the part containing drug. The hole is drilled to accommodate controlled release of drug from the tablet after it is ingested. No disclosure is made of de-massing a tablet via a laser for scoring said tablet to facilitate division of the tablet or any other purpose other than controlling release of drug from the coated tablet. Such controlled release tablets are specifically intended to be taken whole; breaking them would damage the controlled release characteristics created by the coating and laser hole.

U.S. Pat. No. 6,488,192 describes an apparatus that provides half tablets using a cutter for notching a tablet, and a receiver plate and divider roller for dividing the tablet into two equal halves. The cutter for notching the tablet is disposed at the distal end of a reciprocating arm which is moveable vertically and horizontally to position the cutter. The notch provided by the apparatus described in the '192 patent serves solely as a preparation for splitting of a whole tablet into two halves so that half-tablets can be packed and supplied as pre-split half doses that in our terminology would be known as tablettes. The '192 patent is not directed to, and does not disclose or imply, notching or breaking the tablets other than to create half doses. The '192 patent also discloses tablets that are diverted from the cutter and are not notched and are packaged as whole, intact, unmodified tablets. Unlike the apparatus of the '192 patent, the subject invention creates, among other embodiments, tablets that are scored or otherwise “de-massed” but are unbroken.

The subject invention overcomes the disadvantages of current scoring methods by providing a system and method for scoring any face of a tablet, and providing the score or scores in any convenient or technically feasible size, shape, or depth; or position or direction in the tablet. In addition, the subject invention discloses for the first time a method for the use of a laser to remove material from a pharmaceutical tablet to create a score such as a groove, indentation or perforation into the tablet, in such a fashion as to aid any intentional subsequent breaking of a tablet such as by an end user such as a patient. In addition, we disclose the novel use of a laser to modify an uncoated pharmaceutical tablet or a pharmaceutical tablet that is coated with a film coating or other coating that lacks significant rate-modifying characteristics with regard to an active drug contained within said tablet.

### SUMMARY OF THE INVENTION

The subject invention concerns a system and method for removing material from (“de-massing”) any part of a tablet, in any configuration or position, and in any size, shape or depth after the tablet has been compressed or otherwise formed (“preformed”). It is accordingly an object of the invention to provide a system and method for producing a score or other mark in a tablet after the tablet is formed, but before packaging. More specifically, the system includes (a) a device for removing material from the preformed tablet and (b) a conveyer for positioning the preformed tablet and the tablet material removal device in positional relation so that the device can remove material from (“de-mass”) the preformed tablet. Because the device removes mass from the tablet, this device is referred to herein as a “de-massing” device. Preferably, this de-massing device is provided in the system at a particular station, or “de-massing” station.

The method of the subject invention includes the steps of: (a) providing a preformed tablet; (b) positioning the tablet and at least one de-massing device in positional relation to each other to enable formation of a scored tablet surface by the de-massing device; and (c) removing material from the tablet using the de-massing device to form a scored surface in the tablet; and (d) collecting the scored tablet for use, e.g., packaging.

It is another object of the invention to provide a system and method of use of a laser or another energy-emitting apparatus to remove material from a tablet in order to remove mass from (or “de-mass”) the preformed tablet to form a mark, such as a groove or line which serves as a score to facilitate or enhance the breakability of that tablet. One aspect of the subject invention is that is adapted for high speed processing that would be useful in a commercial operation.

The term “de-mass” or “de-massing” as used herein means any intentional removal of material from a preformed tablet in a manufacturing process. In a preferred embodiment, the material is removed from the preformed tablet to form a score line. To “de-mass” includes, without limitation, to: remove material from a preformed tablet to form a design or logo (logotype) in the tablet; “trim” an edge of a tablet; or, remove material to provide a tablet of a predetermined size or mass. “De-mass” or “de-massing” for purposes herein would not refer to an inadvertent or unintentional loss of material that may occur during processing or handling during or after tablet manufacture. For example, accidental chipping of a tablet by a nurse or patient is not part of the subject invention. As an additional example, de-massing per the invention does not encompass unintentional loss of mass after a tablet is ejected from a tablet die and while it is collected before undergoing intentional de-massing (such as production of a score) at a de-massing station.

Tablets, preferably pharmaceutical tablets, produced by the subject system and method, are also included as part of the subject invention. It is preferred that the method and system of the subject invention be applied to pharmaceutical tablets, though the invention is not limited to pharmaceutical tablets.

The system of the subject invention concerns an apparatus or device that removes material from at least one part of a tablet subsequent to tablet formation, e.g., following tablet compression. The system comprises a tablet material removal device (herein referred to as a “de-massing device”) such as a rotating saw blade, fixed knife or other type of cutting blade; a drill or other boring apparatus; a file or rasp; a laser or other energy-emitting source that may de-mass a tablet. As used herein, the term de-mass refers to removal of material or mass from a preformed tablet, such as removal of tablet material to form a linear indentation or groove into the surface of a tablet face, as is commonly understood in the pharmaceutical arts as a score. In addition, for purposes of the subject invention, the term “score” also includes without limitation various types of markings or “etchings” formed into the surface of a tablet, such as a series of intermittently placed perforations, holes, or indentations. In a preferred embodiment of the invention, such a marking or “etching” serves as a conventional score that guides and/or aids optional breaking of the tablet, but is also intended to include any marking or design formed by de-massing. A unique design such as a company logo that is placed appropriately into a part of a tablet that could be so marked during tablet compression may have use in preventing counterfeiting. In addition, de-massing of a tablet according to the invention may have other uses. As suggested above, a de-massing technique may be used to intentionally decrease the mass of a pharmaceutical tablet, either by removing active drug or an inactive section of the tablet; this may be useful in order to cause a production run of pharmaceutical tablets to meet tight specifications on content uniformity in order to be marketed. De-massing is not limited as to the means by which it is performed.

Advantageously, the subject invention provides for formation of a score at any convenient or technically feasible position in a tablet. For example, the subject invention can involve forming a score into a midline of a tablet to facilitate a break substantially along that midline to provide two substantially equal halves. Alternatively, the score can be formed to guide breaking of the tablet into more than two equal or unequal portions, e.g., forming a triangular or “pie-slice”-shaped portion in a tablet. Further, the method and system of the subject invention can provide an eccentric (i.e. not in the midline, or non-bisecting) score whereby a tablet can be divided asymmetrically through said score into two or more unequal portions when the tablet is broken through said score in the usual manner.

A tablet having any score formed in accordance with the subject invention by removal of tablet material following tablet formation, whether broken into portions before or after the collection step in the subject system or method, is considered part of this invention.

Another aspect of the invention involves the creation of a plurality of scores subsequent to tablet formation so that the tablet may optionally be divided through the scores into three or more tablettes.

The system can further comprise a tablet receptacle or holder capable of receiving and holding a preformed tablet oriented as desired for scoring, and a conveyance means for moving the tablet or scoring apparatus into position to carry out scoring of the tablet. The conveyance means can be a conveyer, e.g., a belt-driven conveyer, or a turntable, or the like, as is commonly known in the art for positioning a tablet for carrying out a post-tabletting process. The system of the subject invention may be expanded to also include any or all of the following: a reservoir or hopper for holding a plurality of preformed tablets; a dispensing means for dispensing the plurality of tablets from the hopper for use in the system, e.g., into a tablet receptacle; a printing apparatus for printing indicia onto the surface of the tablet; and, a tablet collection means for collecting tablets after they have been de-massed.

Other adaptations to the system would be understood by persons of ordinary skill in the tabletting art. For example, the system can include a means for removing or eliminating residual tablet material after that material is removed from the tablet according to the invention. Such removal of residual material can be achieved by application of positive air flow or pulses, or negative air flow or pulses, directed toward or away from the tablet so that the residual material is removed or distributed away from the tablet location without disrupting the position of the tablet. Air flow, for example, effected by a vacuum or suction system, can also be used to hold the tablet in place during any step of the subject method, and preferably can be applied to hold the tablet in position during the scoring process.

The method of the invention therefore comprises the steps carried out by the subject system, but is not limited to their operation within said system. The subject method comprises de-massing (removing material from), a preformed tablet to form a scored surface in at least one face of the preformed tablet. Thus, the removal of material can result in, and includes for purposes of the scope of this invention, one or more conventional score line or groove; a series of perforations or indentations; or other modification(s) in the surface of a preformed tablet such as a design or symbol, e.g., a logo which by de-massing is “placed” into a tablet. The marking of a tablet caused by removal of material from the tablet in accordance with the method and system of the subject invention may also be useful for identification purposes, such as an anti-counterfeiting measure or to identify source, e.g., carving of a company or brand name or logo, tablet strength or other identifier, as is conventionally carried out by debossing (stamping or imprinting) during formation of a tablet. Alternatively, the tablet can be de-massed in accordance with the method and system of the subject invention for aesthetic purposes.

It should be understood that the subject invention is not limited by the purpose of the de-massing of or formation of a score in the tablet; it is sufficient to be within the scope of the invention that de-massing or removal of material from the tablet be carried out after tablet formation, and that the removed material does not result in a drug exit hole in a coated, controlled release tablet, which is known. One embodiment of the invention includes removing material from a preformed tablet to form a deep score in said tablet, for example a score that extends about 50% through a dimension of the tablet or about 50% or more through a particular layer or segment of a tablet.

The subject invention can include the steps of providing a reservoir or hopper for holding a plurality of preformed tablets; dispensing the tablets from the reservoir or hopper into individual tablet receptacles or any other type of tablet holder, preferably as part of a carrier link, in which each receptacle is configured to hold a tablet in a desired orientation; and positioning the holder and tablet in relation to a scoring apparatus to provide a desired score in the tablet. In one embodiment, the carrier links are provided on a conveyer whereby the carrier link moves in a first direction to a de-massing station, past the de-massing device, and by operation of the de-massing device producing a score in the tablet, e.g., a single score in the middle portion of said tablet(s). These and other objects, features, and advantages of the invention are apparent from the description and drawings herein.

“Tablet,” when applied to the pharmaceutical field, is defined herein along the lines of the definition found in Merriam-Webster's Medical Dictionary: “A small mass of medicated material,” usually compressed, but also, for example and without limitation, molded. Placebo tablets may be subjected to the procedures of the invention, as well. The term “tablet” therefore is meant to include solid compacted masses in various shapes, including conventional round or capsule-shaped homogeneous tablets as well as tablets with non-conventional shapes or configurations such as layered or segmented tablets as have been disclosed and claimed in published patent applications US 2006/0003000 A1, WO 2005/112897, WO 2005/112870, WO 2005/112898, and WO 2005/112900. In those patent applications, the term “tablette” is used, and describes a useful part of a broken tablet, such as a half or quarter tablet that may serve as a dosage form. The use of the term “tablet” in the subject invention is intended to also encompass the use of the invention to a tablette as well as a preformed unbroken tablet.

It is another object to provide whole, scored tablets made by the above system and method. Before undergoing the procedures of the subject invention, said whole tablets are not limited in any way, such as with regard to size; shape; presence or absence of layers; or presence or absence of scores or other indentation, perforations, or markings.

### BRIEF DESCRIPTION OF THE DRAWINGS

FIG. 1 is a diagrammatic plan view of one embodiment of a system of the invention.

FIG. 2 is a sectional view of the scoring station, including a scoring device, in a system of an embodiment of the subject invention as shown in FIG. 1, sectioned through line A-A of FIG. 1.

FIG. 3 is a sectional view of the scoring station, including a scoring device, in a system of an embodiment of the subject invention as shown in FIG. 1, sectioned through line B-B of FIG. 2.

FIG. 4 is a sectional view of the scoring device in the system of the embodiment shown in FIG. 1, sectioned thorough line C-C of FIG. 3.

FIG. 5 is a sectional view of a tablet scored by the system embodiment of FIG. 1.

FIG. 6 is a sectional view of a tablet scored by another embodiment of the invention, capable of forming two non-bisecting scores in a single face of a tablet.

FIG. 7 is a front elevation view of a rotary cutter of another embodiment of the invention.

FIG. 8 is a sectional view of a tablet scored by the rotary cutter of FIG. 7.

FIG. 9 depicts a tablet with two scores, each created by the method and system of the invention.

FIG. 10 is a sectional view of another embodiment of the invention comprising a laser as a scoring apparatus.

FIG. 11 is a plan view of a tablet scored by the embodiment of FIG. 10 wherein a laser is used to form linearly-aligned perforations in a part of the tablet in order to provide a weakened area to facilitate breaking of the tablet along that line of perforation scoring.

FIG. 12 is a sectional view of another embodiment of the invention comprising a fixed blade as a de-massing device.

FIG. 13 is a sectional view of another embodiment of the invention comprising a rotary blade as a de-massing device.

### DETAILED DESCRIPTION OF PREFERRED EMBODIMENTS

The subject invention concerns a system and method of removing material from (“de-massing”), and thereby scoring or otherwise marking, “etching,” or modifying, previously formed pharmaceutical tablets (herein, “preformed tablets”), which in the most preferred embodiments of the invention are compressed pharmaceutical tablets. The system comprises a material-removing, (“de-massing”) device for forming the score or other desired defect in the tablet following creation of the tablet and ejection from the die or other apparatus in which the tablet was formed. The subject system can be well understood by referring to embodiments of the invention as presented in this disclosure and the accompanying drawings. However, it should be understood that the subject system is not limited by these disclosed embodiments and is intended to include additional embodiments as would be recognized by the ordinarily skilled artisan from the disclosure, including the drawings and accompanying text and description provided herein. Moreover, the method of the subject invention is not limited to being carried out within the disclosed system. The subject invention also includes a tablet in which a score or other de-massing into the surface of the tablet has been produced by the disclosed system and method, as well as any composition that comprises a tablet or tablette of the invention.

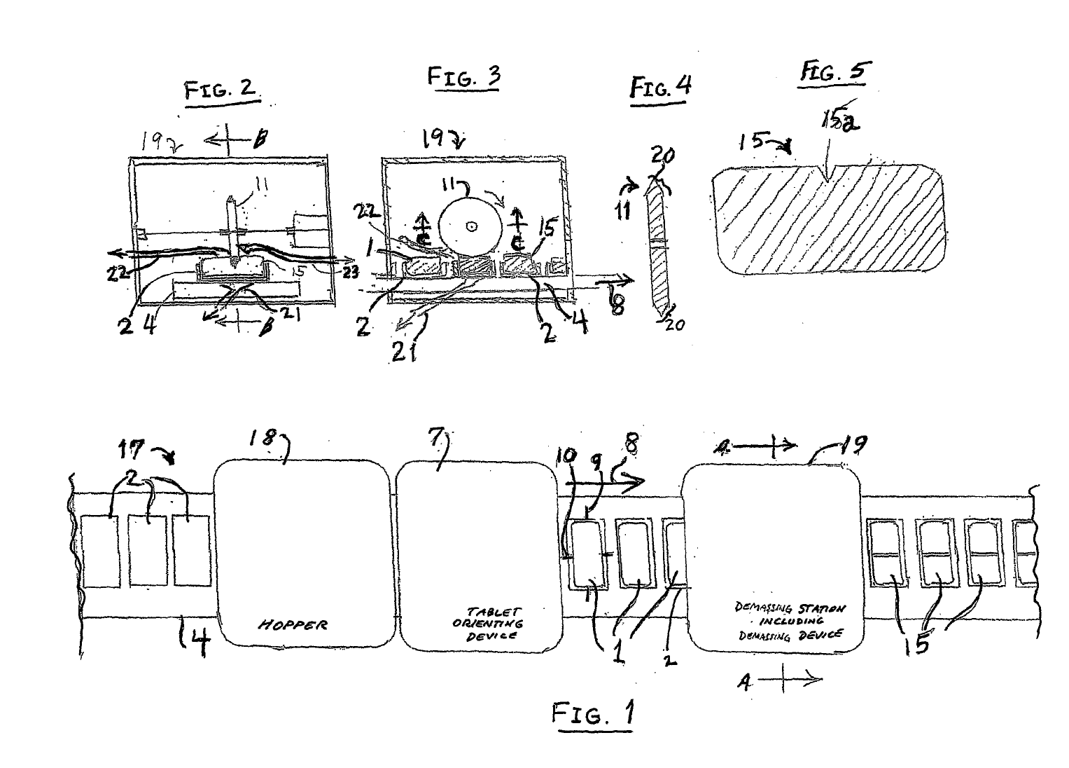

FIGS. 1-4 show an embodiment of a system 17 of the invention. This embodiment comprises a hopper 18 for holding preformed tablets that can be dispensed in a controlled manner for use in the system. Conveyer 4, which in this embodiment moves the tablet from the hopper to a de-massing station 19, can include tablet receptacles 2 which receive and hold tablets during conveyance and scoring. Such a hopper and such a conveyer are well known in the art and can be adapted from a tablet printing system wherein a tablet is marked with indicia following tablet formation. In traveling from the hopper to the de-massing station, the system can also include a tablet orienting device 7 to orient the tablet into a preferred position or orientation for receiving a score. The orienting apparatus 7 orients the individual tablets 1 so that they are held in tablet receptacles 2 wherein the tablet is positioned with one axis aligned parallel with the travel of the carrier links. The orienting apparatus may include rotating wire brushes, deflectors, or the like, as are well known in the art.

The conveyer 4 for moving the tablet from the hopper to the de-massing station 19 (and optionally through the orienting device 7) may involve the use of a rotary drum, an endless belt, or the like, to convey tablets to the de-massing station. The conveyer 4 can carry a plurality of receptacles 2, which are preferably open-top receptacles to facilitate receipt of the tablet. These receptacles 2 are preferably dimensioned so that each will snugly hold a tablet 1 in a particular desired orientation. For example, tablets can be oriented, as shown in this embodiment, with the long axis 9 of the tablet parallel to the direction of movement of the conveyance, and at right angles to the shorter axis 10.

In another embodiment, the receptacles can be open-top and open-bottom providing for access to the tablet by de-massing devices, one positioned above and the other positioned below the tablet on the conveyer.

The conveyer 4 moves in a first direction 8. Preformed tablets in their receptacles are conveyed to a de-massing station 19 where the tablets can be de-massed, e.g., scored, to produce scored tablets 15.

In the de-massing station 19, as illustrated in FIGS. 2 and 3, a motor-driven rotary cutter blade 11 removes tablet material by cutting a slot that serves as a core as the tablet passes under it. A vacuum assembly 21 holds the tablet tightly in the receptacle during removal of tablet material. The cut material may be aspirated by suction lines 22 and 23.

FIG. 4 illustrates an embodiment of blade 11 that can have a contoured edge 20 that produces a correspondingly contoured score 15a in a finished tablet 15 (FIG. 5). In this case, the cross-sectional “V” shape of the score facilitates subsequent optional breaking of the tablet by an end user, nurse, etc. through said score. When a blade of this type was used to score tablets to a depth of 0.043 inches, said tablets were then readily breakable both by hand and with a commercially available tablet splitter.

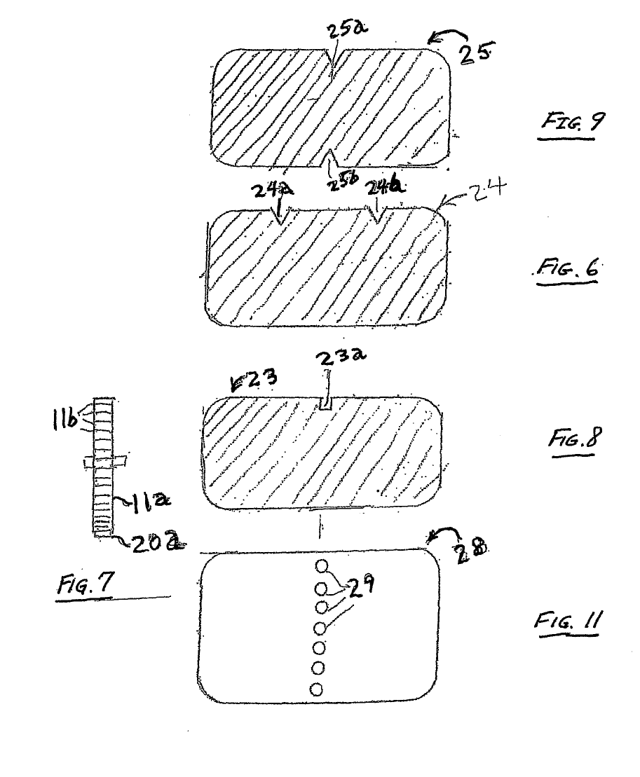

Referring now to FIG. 6, a tablet 24 can include two scores 24a and 24b formed in the same face by the de-massing device so that the tablet may be easily broken into three parts. This dual-scored tablet can be produced by including within the de-massing station two de-massing devices, e.g., rotary cutters, configured to provide two scores parallel to one another. It would be understood that the formation of two scores in the tablet can be achieved by one de-massing device or by scoring a tablet once in one de-massing device and a second time by a second de-massing device.

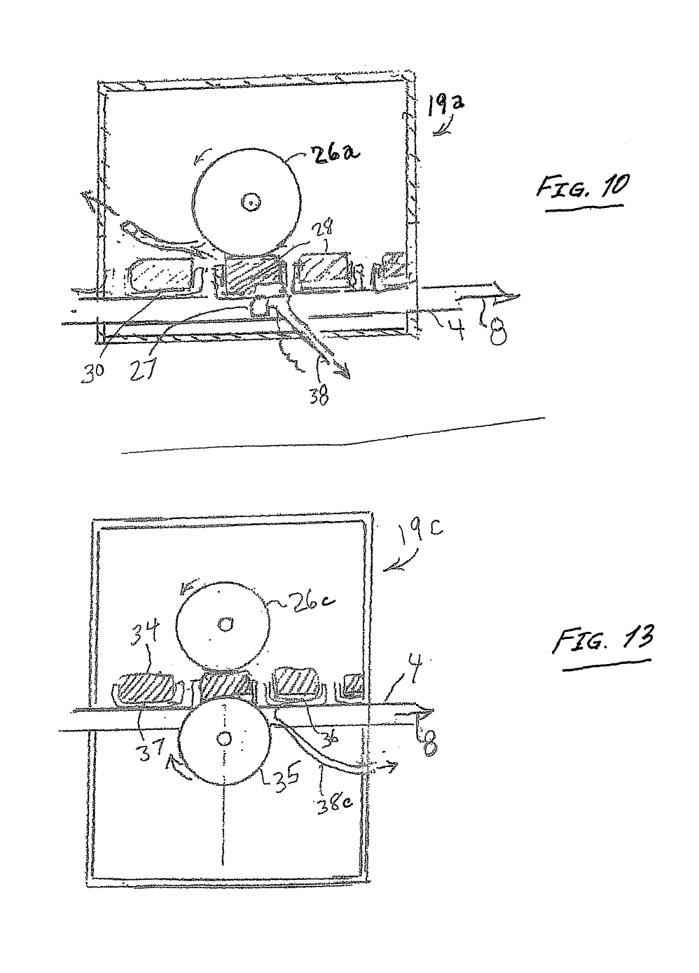

Referring now to FIGS. 7 and 8, the rotary cutter 11a, shown in FIG. 7, may have a squared-off edges (e.g., 20a) to produce the tablet 23 show in FIG. 8 with a squared-off score 23a. The rotary cutter may have teeth 11b as shown (FIG. 7). Alternatively, the rotary cutter may be a wire brush (not shown). No limitations are intended by the above examples.

Referring now to FIG. 9, a tablet may be scored by the invention with two scores 25a and 25b formed on opposite faces of the tablet. By illustration, scores 25a and 25b in tablet 25 may be formed simultaneously by rotary blades of corresponding shape to the score, positioned above and below the receptacle (not shown) or sequentially by flipping the tablet after creation of the first score and subsequently scoring the opposite side.

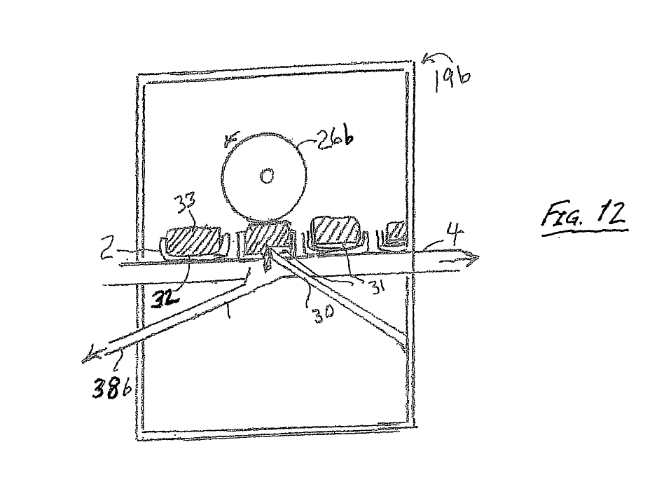

Referring now to FIG. 10, the scoring station 19a comprises a resilient rotary wheel 26 that presses down on the tablet to fix its position and a laser 27 scoring device which can be focused on the underside 30 of the tablet 28 to cut a blind hole on the tablet material. Preferably, the tablet is uncoated. Motion of the tablet on the conveyance 4 rotates wheel 26a due to contact between the tablet and wheel 26a. The resilient rotary wheel 26a can comprise a cushioned material, such as a foam-rubber, sponge-rubber or polymeric material which allows secure positioning of the tablet without crushing the tablet. Materials which perform this function would be recognized and are well know in the art. If the laser is continuously powered as the tablet passes, it produces a slot, forming a score as shown in FIG. 8. Intermittent powering of the laser can produce perforations (or indentations) 29, as illustrated in FIG. 11, which could be utilized instead of a score line to locate a desired tablet breaking region and to weaken the tablet to aid breaking. The holes may be blind holes or they may completely penetrate the tablet. Aspirator 38 of the embodiment shown in FIG. 10 clears away material removed from tablets.

Referring now to FIG. 12, a de-massing station 19b comprises a resilient rotary wheel 26b that presses down on the tablet 33 to fix its position and a scoring device comprising a chisel-shaped fixed cutting blade (knife) 30 which cuts a slot into the tablet, forming score 31 in the underside 32 of the tablet as the tablet passes by the de-massing device on conveyer 4. Motion of the tablet on the conveyer 4 may be used to rotate wheel 26b due to contact between the tablet and wheel 26b. Aspirator 38b clears away material removed from tablets.

Referring now to FIG. 13, scoring station 19c comprises a resilient rotary wheel 26c that presses down on the tablet 34 to fix its position for desired formation of the score, and a scoring device comprising an electric motor-powered rotary cutter (e.g., saw) blade 35 which can cut a score 36 in the underside 37 of the tablet. Motion of the tablet on the conveyer 4 rotates wheel 26c due to contact between the tablet and wheel 26c. Aspirator 38c clears away material removed from tablets.

While we have shown and described certain preferred embodiments of our invention, it will be understood that the invention may be embodied otherwise than as herein specifically illustrated or described, and that certain changes in form and arrangement of parts and the specific manner of practicing the invention may be made within the underlying idea or principles of the invention. For example, all embodiments described herein involve the tablet being held in a tablet receptacle and moved by a conveyer toward a scoring station which comprises a scoring device, e.g., a blade or laser, but it will be readily understood by persons of ordinary skill in the art that the tablets may be held stationary while a scoring device or scoring station is moved in relation to the tablet in order to form a score into the tablet face. It would be further understood that the material-removing device can be moveable, e.g., able to be pivoted.

### MANUFACTURING EXAMPLE

A prototype scoring device was made by modifying a standard R. W. Hartnett Delta tablet/capsule printer. A rotating cutting wheel was added to the standard Delta machine as a scoring device and the conveyer adapted to comprise carrier links, forming tablet receptacles designed to fit and hold the tablets in place during the cutting or scoring operation. The tablets were fed into the carrier links and automatically carried to the cutting wheel de-massing or scoring device by the conveyer adapted from the tablet/capsule printer. The tablets were scored easily by the device. Actual cutting time was much less than one second per tablet. This same machine can print up to 80,000 tablets per hour in its simplest configuration, data which relates to its use absent any modification to score tablets. The scoring device comprises a cutting wheel which can be adjusted in width and to set at a fixed position and score depth into the tablet. The operator turns a set screw to adjust the height of the cutting wheel and therefore the depth of the score. Both the amount of turning and the screw thread determine how far the cutter moves toward or away from the tablet. Placebo tablets were used during the above-described tablet scoring. The tablet dimensions were approximately 8×6×3 mm. The tablets were scored perpendicular to the longest dimension and in the midpoint of said longest axis of the tablet. The first score attempted was not considered to be deep enough because the tablets thereby scored could not be easily split through said score. The score depth in that case was determined to be 0.023 inch deep. The cutter wheel was adjusted so that the cutter blade would penetrate deeper into the tablet. The resulting score depth was determined to be 0.033 inch deep and the tablet was easily breakable manually through said score. The cutter wheel was further adjusted to a depth of 0.043 inches. Tablet breaking through the 0.043 inch score thus formed was easy. When the cutter height was adjusted to be deeper than 0.043 inches, the tablets split in the carrier links during the scoring process.

It is anticipated that for commercial use, the design of a tablet de-massing system may include a sorting and de-dusting station, a hopper for holding bulk preformed tablets for use in the system, a turntable to line up the tablets, and a conveyor belt system to carry the tablets to the de-massing or scoring device and then to a delivery chute and collection bin for collecting said tablets. A vacuum system would likely be installed to reduce local dust accumulation and may be employed to use negative pressure as is known in the art to position the tablet during the cutting or scoring process. A micrometer would likely be added to the scoring adjustment knob that would provide a reading of the depth of the score. The tablet holders or receptacles (carrier links) would be made for different tablet sizes and could be molded to be interchangeable in the conveyer system.

Any suitable tablet, such as a pharmaceutical tablet, may be used. Generally, a powder or granulation may be utilized to form a pharmaceutical tablet, said tablet comprising any suitable active drug with suitable excipients. Molded tablets may be utilized as well, without limiting the invention to compressed or molded tablets. The subject invention may be utilized on pharmaceutical tablets with either immediate release or controlled release characteristics. The nature of the active ingredient is also not limited. Important fields of pharmaceutics suitable for pharmaceutical tablets utilizing the invention include, for example, remedies for cardiovascular disease, diabetes, thyroid disease, coagulation disorders or prothrombotic states, pain, and psychiatric disorders.

The location and direction of any scores created by the methods and systems of the invention are not limited except as may be convenient or technically feasible. Said scoring may be created perpendicular to a theoretical long axis of the tablet and may bisect said theoretical long axis, although some preferred embodiments of the invention involve non-bisecting scoring.

Tablets that are especially suited for the invention comprise tablets involving two or more layers. In a preferred example, a pharmaceutical tablet is produced with an active pharmaceutical agent in a first (bottom) layer and a third (top) layer, with an interposed second (middle) layer formed from an inactive granulation, and the subject invention is used to cut a score into the interposed second (middle) layer without cutting into either the first or third layer (“top,” “bottom” and “middle” relate in this example to the order of entry of said layers into the die). Such a score could not be mass-produced with current tablet-making equipment or with any previously-disclosed apparatus. In another preferred example, a pharmaceutical tablet is created with a first layer comprising an active drug and a second layer comprising only inactive excipients, and said first layer has material removed according to the invention to aid optional tablet breaking such as by an end user such as a patient.

1. A method of removing material from a tablet after the tablet is formed, said method comprising:
a) providing a preformed tablet;
b) positioning said tablet and at least one de-massing device in positional relation to each other to enable formation of a scored tablet surface by said de-massing device;
c) removing material from said tablet using said de-massing device to form a scored surface in said tablet; and,
d) collecting said scored tablet.

2. The method of claim 1 wherein said tablet is a pharmaceutical tablet.

3. The method of claim 1 wherein said positioning step comprises conveying the tablet to the de-massing device to enable removal of tablet material by the de-massing device.

4. The method of claim 1, said method further comprising the step of orienting the tablet in a desired orientation prior to removing material from the tablet.

5. The method of claim 1 wherein said method further comprises the step of dispensing the preformed tablet from a hopper prior to positioning and tablet material removal.

6. The method of claim 1 wherein said material removing step is carried out using a de-massing device selected from the group consisting of a laser, a knife, a drill, and a file or rasp.

7. The method according to claim 1, said method comprising removing tablet material to form a score line for aiding in tablet breaking.

8. The method of claim 7 comprising forming the score line in the underside of the tablet.

9. The method of claim 7 wherein said score is formed in a single segment of a tablet comprising a plurality of segments.

10. The method of claim 1 wherein said removal of tablet material provides at least two scores in the tablet surface.

11. A composition produced by the method of claim 1, said composition selected from the group consisting of a tablet, a pharmaceutical tablet, a tablette, and portions thereof.

12. A system for removing tablet material to provide a scored tablet surface after the tablet is formed, said system comprising:
a) a de-massing station comprising at least one de-massing device for removing tablet material from a preformed tablet, and
b) a conveyer for positioning the preformed tablet and de-massing device in positional relation to enable formation of a scored surface in the tablet.

13. The system according to claim 12 in which said de-massing device comprises cutting instrument selected from the group consisting of a laser, a rotating saw blade, a knife, a drill, and a file or rasp.

14. The system of claim 12 wherein said conveyer comprises a carrier link.

15. The system of claim 12 further comprising a tablet receptacle for receiving and holding said tablet during conveyance and tablet material removal.

16. The system of claim 12 wherein said conveyer carries the preformed tablet to the tablet material removing station for removal of tablet material by the tablet material removing device.

17. The system of claim 12 wherein said system comprises a scoring device as the de-massing device to form a score line in said tablet to aid in tablet breaking.

18. The system of claim 17 wherein said system is adjustable for dimensionally adjusting the score formed by the scoring device.

19. The system according to claim 12 in which a de-massing device is positioned to de-mass an underside of a pharmaceutical tablet.

20. The system of claim 12, said system further comprising a means for removing residual tablet material removed from the tablet by the de-massing device.

21. The system of claim 12, said system further comprising a vacuum aspirator.

22. The system of claim 12, said system further comprising a hopper for holding bulk preformed tablets prior to conveying the tablets to the de-massing station.

23. The system of claim 12, said system further comprising a dispensing means for dispensing preformed tablets from a hopper to a conveyer or tablet receptacle.

24. The system of claim 12, said system further comprising a tablet orienting device to orient the tablet in a desired orientation prior to removal of tablet material by the de-massing device.

25. The system of claim 12, said system further comprising a collection device for collecting scored tablets following formation of a scored surface in the tablet by the de-massing device.

26. A composition produced by the system of claim 12, said composition selected from the group consisting of a tablet, a pharmaceutical tablet, a tablette, and portions thereof.

27. The method of claim 1 wherein said tablet material removal step (c) provides a non-bisecting score in said tablet.

28. The method of claim 27 wherein said tablet with a non-bisecting score is broken along said score into asymmetric portions prior to collection step (d), which comprises collection of tablet portions.

29. The method of claim 1 wherein said tablet material removal step (c) provides a score that is not parallel to a vertical plane of a tablet die.

30. A composition produced by the method of claim 28, said composition selected from the group consisting of a tablet, a pharmaceutical tablet, a tablette, and portions thereof.

31. The system of claim 12 wherein said system further comprises an asymmetric tablet divider for dividing an asymmetrically scored tablet.

32. The method of claim 1 wherein said method includes printing indicia on the tablet prior to the collection step.

33. The system of claim 12 further comprising a printing device to print indicia on the tablet.
